(19) United States
(12) Patent Application Publication
(10) Pub. No.: US 2008/0214075 A1
(43) Pub. Date: Sep. 4, 2008

(54) Finishings For Textile Fibers and Fabrics to Give Hydrophobic Oleophobic and Self-Cleaning Surfaces

(75) Inventors: Oliver Marte, Wattwil (CH); Walter Marte, Ulisbach (CH); Stephan Angehrn, Ulisbach (CH); Martin Meyer, Thalwil (CH); Ulrich Meyer, Zurich (CH); Urs Von Arx, Huttwil (CH); Ruth Weber, Oberburg (CH); Oliver Kunzi, Heimberg (CH); Cedric Clivaz, Niederwangen (CH); Martin Hochstrasser, Neuendorf (CH)

Correspondence Address: SPECKMAN LAW GROUP PLLC, 1201 THIRD AVENUE, SUITE 330, SEATTLE, WA, 98101, US

(73) Assignee: Schoeller Textil AG, Sevelen (CH)

(21) Appl. No.: 11/572,489

(22) PCT Filed: Jul. 18, 2005
(86) PCT No.: PCT/CH05/00420

§ 371 Date: Nov. 27, 2007

(30) Foreign Application Priority Data
Jul. 20, 2004 (CH) 1222/04
Oct. 29, 2004 (CH) 1798/04

(51) Int. Cl. B32B 27/12; C08F 2/46; C08G 65/18; C08J 7/04; C07D 303/40

(52) U.S. Cl. 442/80; 522/168; 522/170; 522/181; 522/182; 522/1; 442/93; 427/513; 118/620

(57) ABSTRACT

Methods for coating fibers and material to achieve particular properties such as water repellency, oil repellency and self-cleaning, are provided. These properties are achieved by pre-treating the fibers or material and subsequently coating with prepolymers and nanoparticles. The prepolymers are partly substituted with hydrocarbon groups or fluorocarbon groups. The nanoparticles may have surfaces treated with hydrocarbons or fluorocarbons. The prepolymers are fixed and hardened by irradiation with light of a wavelength in the UV or visible regions.

This invention relates to finish formulations and finishes on textile fibers and fabrics, as well as to methods for finishing textile fibers and fabrics with such functional layers. In like manner, this invention relates to the textile fibers and fabrics with the finishes according to the invention that were treated or obtained based on the method according to the invention.

The finish formulations and methods currently used in the textile finishing industry are predominantly geared toward fixating the finish formulations, and hence generate an effect permanently at temperatures exceeding 120° C. For example, these include the water repellant finishing and wrinkle-resistant finishing of textiles, as well as the fixation of flame-retardant and softening finishes. Given the prescribed reaction principles that apply to the chemicals used, fixation temperatures of between 140 and 170° C. are absolutely necessary.

The significant disadvantages to this procedure lie in the high energy consumption of the reaction aggregates, the partially inadequate thermal stability of the effect-specific chemicals, as well as their volatility at higher temperatures (evaporation and sublimation), which result in considerable emission problems as an additional shortcoming. The mentioned disadvantages are becoming especially important today in conjunction with the currently required functional finishes for the areas of medicine and wellness, since the functionality of finish components is often lost during their exposure to a thermal load.

In addition, process engineering is today geared toward high fixation temperatures of 120° C. to 180° C., which brings with it operational problems owing to the necessary heating and cooling phases for the fixation aggregates required by changing the article. This results in a machine utilization of only 40 to 70% as a function of article assortment.

These statements show that, in light of the aqueous impregnation and coating liquors used by the textile industry today, new finish formulations and processes are required to enable a permanent fixation of the effect-generating finish formulations already under conditions of drying the impregnated fabric, i.e., at temperatures of below 120° C.

### Radiation-Cured Coatings

Electromagnetic irradiation within the UV range (100-380 nm) and visible range (380 nm-780 nm) makes it possible to polymerize certain monomers and oligomers at room temperature (20° C.). To this end, sensitizers, initiators, coinitiators and accelerators may be necessary. Known radiation-curing monomers and/or oligomers include acrylates, unsaturated polyesters, epoxides, oxetanes and vinyl ether. The acrylates and unsaturated polyester are here polymerized primarily according to a radical reaction mechanism. The epoxides, oxetanes and vinyl ethers primarily follow a cationic reaction mechanism. The chemical principles underlying radiation curing are extensively described in the literature.

One known area of application for radiation-curing monomers and/or oligomers includes dental fillings. Coating glass plates with a monomer mixture of non-fluorinated acrylates and acrylates with perfluorinated side chains followed by UV curing was recently described by Priola et al., UV-curing of Fluorinated Systems: Synthesis and Properties, in: K. Bellfield, J. Crivello, Eds., ACS Symposiums Series 847, Photoinitiated Polymerization, American Chemical Society, Washington, 2003.

A method for imprinting textiles with a UV-curing ink applied by means of an ink-jet process is disclosed in WO-A-0117780. It is printed with a drop volume preferably measuring 75 picoliters, and the UV-light head can be moved over the printed surface together with the printing head or independently thereof.

European Patent Application EP-A-0'120'316 from 1984 describes how the properties of threads and extruded polymer films can be improved by means of coatings. It is proposed that active polyfunctional monomers be used in such a way that neither extensive cross-linking nor grafting essentially takes place. This is intended to help avoid negatively influencing or unintentionally altering the properties of the coated fibers. After the active monomers have been applied, they are dried and cured in a single step via heating or irradiation with an electron beam or UV light. Among other things, the specification proposes that fluoroorganically active monomers be subjected to electron beam curing in order to make polyamide or polyester fibres more hydrophobic and dirt-repellent. While EP-A-0'120'316 does mention that the treatment can also be applied to fabrics, e.g., carpets, the publication does not disclose any specific technical instruction in this regard.

Therefore, the object of this invention is to provide finish formulations, finishes, finished textile fibers and fabrics, and methods for finishing textile fibers and fabrics, that are not associated with the disadvantages to products available on the market and the known devices. In addition, the object of this invention is to provide a type of plant engineering that makes it possible, among other things, to finish and fixate textile fibers and fabrics without the partial loss of the effect-imparting chemicals owing to the currently practiced high-temperature fixation and its energy consumption.

This object is achieved by new finish formulations, new finishing devices and methods, new finishes, and textile fibers and fabrics provided with the new finishes. Combined with the new finishing methods, the specially selected compositions in part new and unusually for the textile industry make it possible to achieve the following, among others:
Monomers or oligomers are 100% cross-linking;
Organic solvent need not be used, so that no VOC (volatile organic carbon) is released during processing and curing;
An emulsion/dispersion in water is possible;
Significantly less energy consumption than in known high-temperature methods;
Short curing time;
Coating of heat-sensitive substrates is possible owing to the low thermal load.

In conjunction with the new type of plant engineering, the formulations and methods according to the invention makes it possible to finish textile fibers and fabrics with UV or blue light-curing chemicals accompanied by a minimal loss in effect-imparting chemicals and a drastically lower energy consumption, and to fixate these chemicals via UV or blue light hardening after evaporating the solvent or dispersive phase.

According to this invention, new finish formulations previously not used in the textile industry along with a type of plant and process engineering are provided that make it possible, parallel to achieving the desired finishing effects, to eliminate high-temperature fixation (140-180° C.) common in the textile industry, thereby lowering the energy required for the finishing process by 40 to 70%. In addition, this technology eliminates the undesired evaporation of the effect-generating chemicals during the fixation process, as well as the resultant necessary investments in an expensive waste air purification system.

The textile fibers or fabrics to be finished consist of native and/or synthetic fibers. After a pre-cleaning stage, they are preferably impregnated with a prepolymer-containing solution, the constitution of which causes it to form a primer layer with reactive groups on the textile article after it has been dried. Prepolymers suitable for primer layers include cross-linkable acrylates, polyamides, urethanes, tannins and lignins. The primer layers applied to the fiber and fabric or generated on the fiber and fabric preferably exhibit a high content of free phenol, hydroxy, amino, carbonic acid or functionally similar groups. Instead of applying the primer layer, the latter can also be generated by treating the base fibers with acids, bases or electrical discharges.

The finish formulation necessary for achieving the desired functionalization of the textile article surface is then applied. This formulation consists of functionalized and non-functionalized comonomers and/or nanoparticles dissolved in solvent or emulsified and/or dispersed in water, the surface of which can also be functionalized. In the case of aqueous systems of the components that functionalize the textile article surface, the formulations preferably encompass surface-active compounds, the constitution of which causes them to lose their function as a surfactant during the fixation reaction, so that they support the functionality desired on the textile article by becoming immobilized and phase-oriented.

The solvent or dispersant is evaporated in a predominantly thermal procedural step that takes place at 60 to 140° C. During this procedural step, the chemicals that functionalize the textile article form the structures necessary to functionalize the textile article via self-organization. The chemical fixation of the completely formed structures takes place in a UV or blue light channel, as opposed to the procedure commonly used in the textile industry today, which provides for thermal treatment to be executed at 140 to 180° C. at this juncture due to different reaction principles for the used chemicals and the lack of plant engineering at the time.

The components of a finish formulation necessary for functionalizing the textile article are reactive monomers or prepolymers that have already been specifically functionalized to reflect the desired effect, and/or nanoparticles, which can in turn also be functionalized and/or contain reactive groups. As additional components, the finish liquor contains surfactants and the dispersant, which in the simplest case is water.

The functionalized monomers or prepolymers are selected based on the target application of the textile article to be functionalized. For example, the textile article is made hydrophobic or oleophobic by means of reactive monomer compounds with correspondingly long hydrocarbon chains or perfluorocarbon chains, which are to be allocated to the acrylates, epoxides, oxetanes, vinyl ethers and mixtures thereof based on their reactive group (hereinafter referred to as “RG”).

During the UV or light curing of acrylates and unsaturated polyesters, which polymerize based on a radical reaction mechanism, curing preferably takes place under a protective gas atmosphere (e.g., nitrogen, CO₂ or argon), since tests have shown that oxygen has a disruptive effect on radical curing, and methods that involve curing without protective gas yield tacky products.

The UV radiators that can be used in this invention can be the UVAPRINT devices made by Dr. Hönle AG, UV-Technologie, Gräfelfing, Germany, for example. According to the manufacturer, the radiator power is usually given in W/cm arc length in these devices. The latter measures a minimum of 30 W/cm and a maximum of 240 W/cm in the preferred devices, most preferably 100 W/cm.

Depending on the desired target applications for the textile article, both inorganic and organic nano-particles can be used as the nano-particles. Typical inorganic nano-particles include silicone dioxide, metal oxides like vanadium, iron, tungsten, titanium, aluminum or zinc oxides, carbon, zeolites, etc. Typical organic nano-particles include target application-oriented, modified dendrimers, glucans or cyclodextrins, which in a preferred embodiment contain metal atoms in complexed form.

The nano-particles can be surface-modified. Typical functional modifying groups that do not undergo further cross-linking include hydrocarbon chains with C₁ to C₂₀ and perfluorocarbon chains with C₁ to C₁₂ and copolymerizable groups like acryl or epoxy groups. Typical cross-linking modifying groups include ethylene oxide or propylene oxide block polymerizates or polyamides, which contain terminal OH- or NH₂-groups, or silanes containing terminal acryl or epoxy groups.

In the case of a finish formulation for hydrophobic and oleophobic layers, the effect-generating groups correspond to the groups described below for monomers (RFᵢ). The monomer units can exhibit both one to three functional groups, generally referred to as “RFᵢ”, as well as several polymerizable groups (RGⱼ). Typical monomer types and ones employed for imparting hydrophobic and oleophobic properties include:

(RGⱼ)ₙ-(CH₂)ₘ—(RFᵢ)ₗ;

(RGⱼ)ₙ-(CH₂)ₘ—N—(RFᵢ)₂;

(RGⱼ)ₙ-(CH₂)ₘ—Si—(RFᵢ)₃

where n is preferably 1 to 3, and m is preferably 0 to 5.

The effect-generating groups (RFᵢ) in the case of hydrophobic and oleophobic layers are hydrocarbons with a chain length of C₁ to C₂₀ and/or perfluorohydrocarbon chains with a chain length of C₁ to C₁₂.

Surfactants include reactive group-containing monomers and/or polymers, the HLB value for which lies between 3 and 16, preferably between 8 and 12.

Typical surfactants include sorbitan laurate or stearate, mono- and diglycerides, ethoxylated and/or propoxylated C₈ to C₂₀ compounds or vinyl or allyl-ether alkoxylates with 10 to 30 EO units, which form addition or condensation products with predominantly nucleophilic reactive groups, e.g., amino and hydroxyl functions.

In the case of wellness finishes, the host system for incorporating wellness substances (guests or drugs) consists of thermally and UV or blue-light curing prepolymers or monomers or UV or blue-light curing prepolymers or monomers, as well as at least one component with a spacer function and a surfactant. A host system structured in this way can be swelled by aqueous emulsions containing at least one guest substance, and are able to sorb and again release the drugs contained in the emulsions.

UV and blue-light curing components essentially include the same compounds as described for use in hydrophobic finishes. The same holds true for the surfactants. The spacer substances that help determine the swelling of the wellness layer are general in type: RG-RS-RG. RG is a UV or blue-light curing reactive group or a functional group that cross-links with such a reactive group, and RS is a residue that characterizes the spacer substance, e.g., a polyether, polyester or vinylog chain:

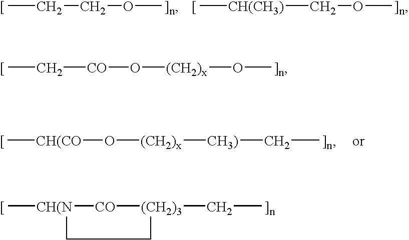

The chain length of the residual RS determining the hydrophilia or hydrophobia of the spacer substance is defined by n and x, wherein n is preferably greater than 5 and less than 30, and x preferably lies between 2 and 4.

### EXAMPLE 1

### Imparting Hydrophilic and Oleophobic Properties to Polyamide Fabrics

A pre-cleaned, dyed polyamide fabric with a square meter weight of 350 g/m² is impregnated with a substance that forms the primer layer in a first procedural step. A copolymer consisting of a partially saponified vinyl acetate and vinyl pyrrolidone is used as the primer substance. The impregnated and dried fabric is impregnated with an aqueous hydrophobic and oleophobic property-imparting emulsion in a second step on a tentering frame foulard. The manufacture and composition of the emulsion will be described below.

The emulsion contains a perfluorinated acrylate, and is manufactured with the following components:

| Component | % w/w |
|---|---|
| Water | 92.75 |
| Emulsogen R109 | 1.45 |
| Laromer BDDA | 5.39 |
| 2-(perfluorodecyl)ethyl-methacrylate | 0.11 |
| 2-hydroxy-2-methyl-1-phenyl-1-propanon | 0.29 |

Emulsogen R109 is a vinyl-ether-alkoxylate with 10 EO made by Clariant.
Laromer BDDA is a butane diol diacrylate made by BASF.

Water and Emulsogen R109 are thoroughly mixed together. The homogenous solution of Laromer BDDA, 2-(perfluorodecyl)ethyl-methacrylate and 2-hydroxy-2-methyl-1-phenyl-1-propanon is added to this mixture in small portions.

The fabric impregnated with a liquor application of 80% relative to the dry weight of the textile article on the tentering frame foulard is subsequently dried at 120° C. for 2 minutes and, after dried passes through a UV channel to fixate the hydrophobic and oleophobic property-imparting layer. The reaction time in the UV channel measures 2.5 seconds at a specific radiated power of 5.5 kW/m². The UV channel is flushed with nitrogen or some other inert gas, such as CO₂ or argon, in order to avoid any undesired oxidation processes on the one hand, and prevent ozone formation on the other.

The fabric finish manufactured in this way is distinguished by a drastic reduction in energy costs as compared with the fixation processes in practice today on the one hand, while outstanding use properties, e.g., contact angle of 137 to 147° and water spray notes of 5, are achieved on the other.

### EXAMPLE 2

### Imparting Hydrophobic and Oleophobic Properties to a Mixed Fabric

A mixed fabric pre-cleaned with solvent, consisting of 94% nylon 6.6 and 6% elastane and having a run meter weight of 280 g/m, is dried after dyed (as part of a 4 hour dying process) and provided with a primer layer. The primer substance is a lignin product, which is applied to the textile substrate as a 0.5% aqueous solution. After drying the fabric at 130° C. for 90 seconds, the hydrophobic or oleophobic property-imparting emulsion is applied. The formulation recipe and its manufacture are described below.

The emulsion contains a C18 acrylate, and is manufactured with the following components:

| Component | % w/w |
|---|---|
| Water | 92.96 |
| Emulsogen R109 | 1.41 |
| OTA 480 | 3.10 |
| Methacrylic acid-octadecyl ester | 2.25 |
| 2-hydroxy-2-methyl-1-phenyl-1-propanon | 0.28 |

OTA 480 is propoxylated trimethylol propane-triacrylate made by UCB.

Water and Emulsogen R109 are thoroughly mixed together. The homogenous solution of OTA 480, methacrylic acid-octadecyl ester 2-hydroxy-2-methyl-1-phenyl-1-propanon is added to this mixture in small portions.

Following the application of a 72% coat of finish liquor, the textile article is secured in the tentering frame and dried for 90 seconds at 120° C. Since the nano-scale structures required for imparting hydrophobic and oleophobic properties (orientation of the fatty and/or perfluorinated hydrocarbon residues relative to the adjacent gas phase) are completely formed after the fabric has been dried, the fabric can be intermediately stored in a dry space. The layer is UV fixated independently of the manufacture of the functional layer, as opposed to the currently usual procedure, in which the dried, hot fabric (120-140° C.) is introduced directly into the condensation phase (150-180° C.), for energy-related reasons as well. A reaction time of 0.5 to 10 seconds at a radiated power of 5.5 kW/m² is necessary for fixation. The contact angles achieved with this finish measure 128-132°, and also exhibit a spray note of 5, even after five washes.

### EXAMPLE 3

### Imparting of Hydrophobic and Oleophobic Properties to Polyamide Fabrics with Self-Cleaning Properties

A pre-cleaned dyed polyamide fabric with a square meter weight of 350 g/m² is subjected to tannin impregnation in a second procedural step to improve colorfastness. The impregnated and dried fabric is impregnated with an aqueous hydrophobic and oleophobic property-imparting, self-cleaning emulsion in a second step on a tentering frame foulard. The manufacture and composition of the emulsion will be described below.

The emulsion contains a perfluorinated acrylate and perfluorinated nanoparticle, and is manufactured with the following components:

| Component | % w/w |
|---|---|
| Water | 92.49 |
| Emulsogen R307 | 1.45 |
| SR350 | 5.39 |
| 2-(perfluorodecyl)ethyl-methacrylate | 0.11 |
| 2-hydroxy-2-methyl-1-phenyl-1-propanon | 0.29 |
| SiO2 perfluorooctyl nanoparticles, 12 nm | 0.27 |

Emulsogen RAL307 is an allyl-ether-alkoxylate with 30 EO made by Clariant.
SR350 is a trimethylol propane-trimethacrylate made by Sartomer.

Water, Emulsogen R307 and nanoparticles are thoroughly mixed together. The homogenous solution of SR350, 2-(perfluorodecyl)ethyl-methacrylate and 2-hydroxy-2-methyl-1-phenyl-1-propanon is added to this mixture in small portions.

The fabric impregnated with a liquor application of 80% relative to the dry weight of the textile article on the tentering frame foulard is subsequently dried at 120° C. for 2 minutes, and, after dried passes through a UV channel to fixate the hydrophobic and oleophobic property-imparting layer. The reaction time in the UV channel measures 2.5 seconds at a specific radiated power of 5.5 kW/m². The UV channel can be flushed with nitrogen in order to avoid any undesired oxidation processes on the one hand, and prevent ozone formation on the other.

The fabric finish manufactured in this way is distinguished by a drastic reduction in energy costs as compared with the fixation processes in practice today on the one hand, while outstanding use properties, e.g., contact angle of 150° and water spray notes of 5, are achieved on the other.

### EXAMPLE 4

### UV-Curable Finish Formulation Containing an Epoxide Monomer and an Oxetane Monomer, as well as an Epoxide Monomer with Perfluorinated Residue

| Component | % w/w |
|---|---|
| Water | 92.52 |
| UVR 6105 | 2.80 |
| UVR 600 | 1.40 |
| 3-(1H,1H,9H-hexadecafluorononyloxy)- | 1.12 |
| 1,2-epoxypropane | |
| UVI 6992 | 0.56 |
| Span 60 | 0.08 |
| Montanox 60 DF | 0.20 |
| Ethanol | 1.32 |

UVR 6105 is an epoxy resin sold by Dow.
UVR 6000 refers to an oxetane resin from Dow.
UVI 6992 denotes a photoinitiator, comprised of a triarylsulfonium hexafluorophosphate salt sold by Dow.
Span 60 is a sorbitan monostearate made by Uniqema.
Montanox 60 DF refers to sorbitan monostearate made by TensoChema AG.

Span 60 and Montanox 60 DF are mixed together in a first step. A previously fabricated mixture of UVR 6105, UVR 600, 3-(1H, 1H, 9H-hexadecafluorononyloxy)-1,2-epoxypropane and UVI 6992 is then added while thoroughly blending. The 3-(1H,1H,9H-hexadecafluorononyloxy)-1,2-epoxypropane is here present as a solution in ethanol. The water is added to the resulting mixture in small portions and while blending thoroughly.

### EXAMPLE 5

### UV-Curable Finish Formulation Containing an Epoxide Monomer and an Epoxide Monomer with C₁₈ Residue

| Component | % w/w |
|---|---|
| Water | 90.62 |
| UVR 6105 | 3.30 |
| 1,2 octadecenoxide | 1.92 |
| UVI 6992 | 0.55 |
| Montane 80 VG | 0.08 |
| Tween 80 | 0.19 |
| Ethanol | 3.34 |

Montane 80 VG refers to a sorbitan monooleate made by TensoChema AG.
Tween 80 denotes a sorbitan monooleate made by Fluka.

Montane 80 VG and Tween 80 are mixed together in a first step. A previously fabricated mixture of UVR 6105, 1,2 octadecenoxide, UV 6992 and ethanol is then added while thoroughly blending. The water is added to the resulting mixture in small portions and while blending thoroughly.

### EXAMPLE 6

### UV-Curable Finish Formulation Containing an Epoxide Monomer and an Epoxide Monomer with a C₁₈ Residue

| Component | % w/w |
|---|---|
| Water | 93.75 |
| OXT-121 | 3.89 |
| UVR 600 | 1.42 |
| IRGACURE 250 | 0.28 |
| 3-(perfluoro-n-decyl)-1,2-propenoxide | 0.09 |
| Synperonic PE/F 108 | 0.57 |

OXT-121 is an oxetane resin sold by Toagosei.

IRGACURE® 250 refers to a cationic photoinitiator consisting of an iodonium salt, (4-methylphenyl) [4-(2-methylpropyl)phenyl]-iodonium-hexafluorophosphate sold by Ciba. Synperonic PE/F 108 is a vinyletheralkoxylate (approx. 14,000 g/mol) made by Uniqema.

Synperonic PE/F 108 and a previously fabricated mixture of OXT-121, UVR 6000, 3-(perfluoro-n-decyl)-1,2-propenoxide and IRGACURE 250 are mixed together. The water is added to the resulting mixture in small portions and while blending thoroughly.

### EXAMPLE 7

### UV and Blue Light Curable Finish Formulation Containing an Oxetane Monomer and an Epoxide Monomer with a C₁₈ Residue

| Component | % w/w |
|---|---|
| Water | 92.96 |
| OXT-121 | 3.10 |
| IRGACURE 250 | 0.28 |
| 1,2 octadecenoxide | 2.25 |
| Synperonic PE/F 108 | 1.41 |

Synperonic PE/F 108 and a previously fabricated mixture of OXT-121, 1,2 octadecenoxide and IRGACURE 250 are mixed together. The water is added to the resulting mixture in small portions and while blending thoroughly.

### EXAMPLE 8

### Wellness Finish for a Polyamide Fabric

A pre-cleaned dyed polyamide fabric with a square meter weight of 180 g/m² is impregnated with a solution of 5 g/l Rewin RT (BEZEMA AG) to improve colorfastness. The pretreated and dried fabric is impregnated with an aqueous wellness emulsion in a second step on a tentering frame foulard. The manufacture and composition of the emulsion will be described below.

The emulsion is manufactured with the following components:

| Component | % w/w |
|---|---|
| Water | 93.0 |
| Superonic PE/F108 | 1.40 |
| OTA 480 | 2.10 |
| UVR 6105 | 1.61 |
| Pluronic PE 6200 | 1.05 |
| Ethylhydroxyethyl cellulose | 0.21 |
| Sorbitan monolaurate | 0.35 |
| 2-hydroxy-2-methyl-1-phenyl-1-propanon | 0.28 |

Superonic PE/F108 refers to a vinyl ether alkoxylate (approx. 14,000 g/mol) made by Unicema.
UVR 6105 is an epoxy resin sold by Dow.

Water and Superonic PE/F 108 are thoroughly mixed together. The mixture of OTA 480, UVR 6105, Pluronic PE 6200, ethylhydroxyethyl cellulose, sorbitan monolaurate and 2-hydroxy-2-methyl-1-phenyl-1-propanon is added to this mixture in small portions.

The fabric impregnated with a liquor application of 80% relative to the dry weight of the textile article on the tentering frame foulard is subsequently dried at 120° C. for 2 minutes, and, after dried passes through a UV channel to fixate the wellness layer. The reaction time in the UV channel measures 2.5 seconds at a specific radiated power of 5.5 kW/m². The UV channel can be flushed with nitrogen in order to avoid any undesired oxidation processes on the one hand, and prevent ozone formation on the other.

The fabric finish manufactured in this way is distinguished by excellent host properties, which in turn are characterized by the good swelling capability of the host layer and the high affinity to lipophilic substances. The layer fabricated in the described manner exhibits a specific substance absorption of 23 mg isooctanol (model substance for therapeutic and/or cosmetic active substances) per gram of host layer. Another essential host property criterion is the reloading of the host layer after the respective article of clothing has been washed. The reloadability of the finish layer still measures 82% of the original sorption capacity for isooctanol after five washes. In addition to the properties relating to the functionality of the finish layers, mention must be made of their cost-effective manufacture, since the high-temperature fixation that normally takes place is omitted.

1: A finish formulation for manufacturing finishes on textile fibers and fabrics comprising:
(a) a first component selected from the group consisting of: functionalized monomers, prepolymers, functionalized-group carrying nanoparticles and reactive-group carrying nanoparticles;
(b) at least one surfactant; and
(c) at least one second component selected from the group consisting of: dispersants and solvents,
wherein the first component can be polymerized by light in the UV range of 100 nm to 400 nm, or in the visible range of 400 nm to 800 nm.

2: The finish formulation according to claim 1, wherein the first component is a functionalized monomers or prepolymers that includes at least one free acrylate, epoxide, oxetane or vinyl ether groups.

3: The finish formulation according to claim 1 wherein the first component is a monomer having at least one effect-generating group comprising one to three functional groups (RFᵢ) and one or more polymerizable groups (RGⱼ).

4: The finish formulation according to claim 3, wherein the monomer has a formula selected from the group consisting of:
(RGⱼ)ₙ-(CH₂)ₘ—(RFᵢ)ₗ;
(RGⱼ)ₙ-(CH₂)ₘ—N—(RFᵢ)₂; and
(RGⱼ)ₙ-(CH₂)ₘ—Si—(RFᵢ)₃
where n is 1 to 3, and m is 0 to 5.

5: The finish formulation according to claim 4, wherein the effect-generating groups (RFᵢ) comprise hydrocarbons with a chain length of C₁ to C₂₀ and/or fluorocarbons with a chain length of C₁ to C₁₂.

6: The finish formulation according to claim 1, wherein the at least one surfactant comprises monomers and/or polymers with an HLB value of between 3 and 16.

7: The finish formulation according to claim 6, wherein the at least one surfactant is selected from the group consisting of: sorbitan laurate, sorbitan stearate, mono- and diglycerides, ethoxylated C₈ to C₂₀ compounds, propoxylated C₈ to C₂₀ compounds, and vinyl or allyl-ether alkoxylates with 10 to 30 EO units.

8: The finish formulation according to claim 7, wherein the surfactants form addition or condensation products with predominantly nucleophilic reactive groups.

9: The finish formulation according to claim 1 for manufacturing a reloadable and swellable finish layer, the finish formulation comprises at least one spacer substance of a general type (RG-RS-RG), wherein RG is a UV or blue-light curing reactive group, or a functional group that cross links with such a reactive group, and RS is the residue characterizing the spacer substance.

10: The finish formulation according to claim 9, wherein the residue that characterizes the spacer substance is selected from the group consisting of a polyether, polyester or vinylog chain, with the formula: 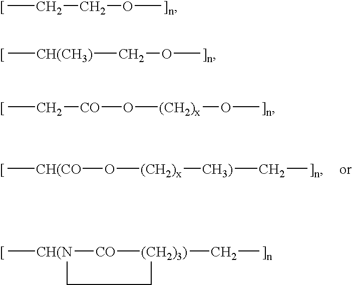
wherein the chain length of the residual RS is defined by n and x, wherein n is greater than 5 and less than 30, and x between 2 and 4.

11: The finish formulation according to claim 1, wherein the nanoparticles comprise inorganic and/or organic nanoparticles selected from the group consisting of: silicone dioxide, metal oxides, carbon, zeolites, dendrimers, glucans and cyclodextrins.

12: The finish formulation according to claim 11, wherein the nanoparticles on the surface comprise hydrocarbon chains with C₁ to C₂₀ or fluorocarbon chains with C₁ to C₁₂, and copolymerizable groups.

13: A finish on textile fibers and fabrics for achieving hydrophobic, oleophobic and/or self-cleaning surfaces on a textile article, wherein the finish is manufactured with the finish formulations according to claim 1.

14: A finish according to claim 13, wherein the functionalizing chemicals form the structures necessary to functionalize the textile article via self-organization.

15: A finish according to claim 13 wherein the finish is manufactured with finish formulations according to claim 9, can be swelled by aqueous emulsions, and is able to sorb active substances contained in the emulsion.

16: A method for manufacturing finishes for textile fibers and fabrics, wherein at least one finish formulation according to claim 1 is used.

17: The method according to claim 16, wherein the at least one finish formulation is applied to the textile fibers and fabrics, dried at about 60 to 140° C., and fixated by light in the UV range of about 100 nm to 400 nm, or in the visible range of about 400 nm to 800 nm.

18: The method according to claim 17, wherein irradiation for fixation takes between 0.5 and 10 seconds.

19: The method according to claim 17 wherein irradiation takes place at a specific radiation intensity of 5.5 kW/m².

20: The method according to claim 17, wherein a primer layer with reactive groups is applied before applying the at least one finish formulation.

21: The method according to claim 20, wherein the primer layer comprises at least one cross-linkable prepolymers selected from the group consisting of: acrylates, polyamides, urethanes, tannins and lignins.

22: The method according to claim 20, wherein the primer layer has a high content of free phenol, hydroxy, amino and/or carboxylic acid groups.

23: The method according to claim 16, wherein solvent or dispersant is evaporated while drying, and wherein the chemicals that functionalize the textile article form the necessary structures to functionalize the textile article via self-organization.

24: The method according to claim 16, wherein acrylates and unsaturated polyesters, which polymerize based on a radical reaction mechanism, are cured with light in the UV range or visible range.

25: A fiber or fabric having a finish according to claim 13.

26: An engineering plant for implementing methods for manufacturing finishes for textile fibers and fabrics, wherein the engineering plant comprises a devices for applying a finish formulation to the textile fabric, a drying device for drying at about 60 to 140° C., and a curing device with illuminating means for generating radiation in a UV range of about 100 m to 400 nm, or in the visible range of about 400 nm to 800 nm.

27: An engineering plant according to claim 26, wherein the curing device comprises a UV channel with illuminating means at radiator powers in W/cm arc length of 30 W/cm to 240 W/cm.

28: An engineering plant according to claim 27, wherein the UV channel has means for flushing with a protective gas.
